(19) United States
(12) Patent Application Publication
(10) Pub. No.: US 2007/0208303 A1
(43) Pub. Date: Sep. 6, 2007

(54) Syringe of a Non-Reusable Type

(76) Inventor: Gudmar Olovson, Paris (FR)

Correspondence Address: SEYFARTH SHAW LLP, 131 S. DEARBORN ST., SUITE2400, CHICAGO, IL, 60603-5803, US

(21) Appl. No.: 10/593,849

(22) PCT Filed: Mar. 23, 2005
(86) PCT No.: PCT/SE05/00420

§ 371 Date: Sep. 22, 2006

(30) Foreign Application Priority Data
Mar. 23, 2004 (SE) 0400736-5

(51) Int. Cl. A61M 5/50

(52) U.S. Cl. 604/110

(57) ABSTRACT

The present invention encompasses a syringe of non-reusable type comprising: a container, a rod cooperating with the container, a piston unit inserted in and reciprocally disposed in the container, and a needle, the rod being, by an axial displacement movement, moveably disposed reciprocally in said container and displaying in its one end portion enclosed by the container a first coupling device within a two-part coupling arrangement whose second coupling device is related to said piston unit. Said two coupling devices assume a mutually cooperating and active position while the piston unit is, by the movement of the rod, displaced from a position closely adjacent the needle to a position distal from the needle and causes said coupling devices to be gradually brought to an inactive position while the piston unit is, by the movement of the rod, displaced from the distal position in relation to the needle towards and/or to the position closely adjacent the needle, the two coupling devices permitting, in an inactive position, an axial movement of the rod to take place without cooperation with the piston unit. Said second coupling device is provided with means which is rotatably coordinated with a piston included in said piston unit.

### TECHNICAL FIELD

The present invention relates generally to a syringe of so-called non-reusable type.

Syringes or injection syringes of non-reusable type are previously known in numerous versions and embodiments and these are adapted and intended to be able to be used once only, i.e. a container must be capable of being filled with a liquid when displacement of a rod associated with the syringe in cooperation with a piston associated with the syringe takes place from a fully protracted position to a fully retracted position.

A two-part coupling arrangement disposed between the rod and the piston is intended during this displacement movement to assume an “active” position by which is intended that a coupling device associated with the rod is in a selected mode of cooperation with a coupling device associated with the piston.

On an opposing displacement movement of the rod and the piston, the two-part coupling arrangement is brought with its coupling devices to an “inactive” position.

The present invention relates more specifically to such an injection syringe of non-reusable type as includes a container, a rod cooperating with the container, a piston unit inserted in the container and reciprocal by means of the movement of the rod, and a cannula or needle, the rod being, without rotational movement in relation to the container, axially displaceable reciprocally in said container and displaying, in its one end region enclosed by the container, a first coupling device included in the two-part coupling arrangement, whose second coupling device is related to said piston unit.

Said two coupling devices, within said two-part coupling arrangement are then to assume a mutually cooperating and active position, while the piston unit is displaced by the movement of the rod from a position closely adjacent the needle to a position distal from the needle and thereafter said cooperating coupling devices are gradually brought to a completely inactive position during a counter-directed movement of the rod.

On a renewed axial displacement of the rod from the position closely adjacent the needle, this displacement will take place without cooperation with the piston unit and the piston which will be located in a position closely adjacent the needle and, thereby, the injection syringe will be unusable.

### BACKGROUND OF THE INVENTION

A plurality of different embodiments of methods and arrangements of the above disclosed nature are previously known in the art.

Injection syringes of the non-reusable type and with a two-part coupling arrangement active between the rod and the piston in the manner described above may be divided into two categories, namely:
a. With a rod inserted in the container and to which is imparted a rotational movement during its displacement movement; and
b. with a rod inserted in the container and to which no rotational movement is imparted during its displacement movement, hereafter referred to as “axial displacement movement”.

As regards the prior art which falls within the category “a” above, mention might be made of the following.

As a first example, reference is made to the contents of Swedish Patent Specification SE-B-469 263 (Patent Application Number 91 01204-7) which discloses and describes a piston (4) included in a syringe intended for injecting a liquid, and consisting of a container (2), a cannula or needle (3), said piston (4) and a rod (5) as well as means interconnecting and disconnecting the piston and the rod, or a two-part coupling arrangement (6).

Said means (6) assumes an interconnecting state when the piston is drawn by the rod from a position closely adjacent the needle (3) to a position distal from the needle, and is brought to a disengaging state when the piston (4) has been urged by the rod in a direction towards the needle.

Said rod (5) is disposed to cooperate with the container (2) in such a manner that, on a displacement in relation to the container (2) a rotational movement is imparted to it in relation to the container.

The portion of the rod (5) facing towards the needle (3) is formed with support and/or sliding surfaces (5k, 5k′) which are disposed at right angles or in any event substantially at right angles in relation to a centre line to the rod (5).

Said surfaces are disposed to cooperate with support and/or sliding surfaces (4k, 4k′″, 4m, 4m′) associated with the piston (4).

Support and/or sliding surfaces associated with the rod (5) and support and/or sliding surfaces (4k, 4k′) associated with the piston (4) are formed peripherally and are disposed to incline somewhat in a direction inclining towards the direction of movement of the rod (5) and opposing peripheral surfaces associated with the piston are formed in peripheral wall portions.

As a further example of the state of the art in this instance, reference is made to the contents of Swedish Patent Specification SE-B-469 264 (Patent Application Number 91 01205-4) which shows and discloses a rod (5) included in a syringe intended for injection of a liquid and consisting of a container (2), a needle (3), a piston (4) and said rod (5), as well as means interconnecting and disconnecting the piston (4) with the rod (5) or a two-part coupling arrangement (6).

Said means assumes an interconnecting state when the piston is drawn by the rod from a position closely adjacent the needle (3) to a distal position in relation to the needle, and is brought to a disconnecting state when the piston is urged by the rod in a direction towards the needle.

Said rod (5) is disposed to cooperate with the container in such a manner that, on a displacement in relation to the container (2) a rotational movement is imparted to it in relation to the container (2) in that a rotation of 90° or thereabouts is imparted to the rod in its longitudinal extent.

The portion of the rod facing towards the needle is formed with support and/or sliding surfaces disposed to cooperate with opposing support and/or sliding surfaces associated with the piston. In this instance, it is particularly disclosed that the rod displays a right-angle cross section and is dimensioned with a broad central thin portion (5p).

The contents of Swedish Patent Specification SE-B-79 02138-2 carrying Publication Number 438 598 also forms part of the state of the art.

As regards the prior art falling within category “b” above, mention might be made of the following.

One such construction of an injection syringe of non-reusable type is shown and described in Swedish Patent Specification SE-C2-504 013 (Patent Application Number 95 02697-7) which shows and describes a syringe (1) of non-reusable type, including a rod (2) which is reciprocally disposed in relation to a container (3) whose one end (31) consists of, or may cooperate with, a needle (11) and whose opposing end (32) is open, a piston (12) cooperating with the end of the rod facing into the container, and means interconnecting and disconnecting the piston with the end of the rod, or a two-part coupling arrangement (13).

In such instance, reference is made to two edge-related guide and sliding surfaces (22a, 22b) associated with the rod and oriented in its longitudinal direction.

Said interconnecting and disconnecting means (13) is to assume an interconnecting state when the piston (12) is drawn by the rod (2) from a position closely adjacent the needle (11) to a position distal from the needle, and is brought to a disconnecting or disconnected state when the piston is urged by the rod in a direction towards the needle.

The support and/or sliding surfaces of the rod associated with the portion (21) facing towards the needle are disposed to cooperate with opposing support and/or sliding surfaces associated with the piston (12) while a rotation of said rod about a common centre axis (A) to the rod and the container is prevented in that controlling means (4) associated with the container prevent said rotation of the rod. In this construction, a rotational movement will be imparted to the piston against the action of frictional forces active between the piston and the inner surface of the container.

Said controlling means (4) consists of in any event two depressions (41, 42) in an outer circumferential surface (33) associated with the container (3).

Each depression (41, 42) forms a guide- and sliding surface (41a, 42a) in an inner circumferential surface (34) associated with said container and guide- and sliding surfaces (22a, 22b) associated with said rod (2) are disposed to run towards or adjacent and in relation to guide- and sliding surfaces (41a, 42a) associated with said container (3).

### OUTLINE OF THE PRESENT INVENTION

Problem Structure

Considering the circumstance that the technical deliberations that must be made by a person skilled in the art to be able to offer a solution to one or more technical problems posed is, on the one hand, initially a necessary insight into the measures and/or sequence of measures to be adopted and, on the other hand, a necessary selection of the means required, the following technical problems are likely, in view hereof, to be relevant in the evolution of the subject matter of the present invention.

Considering the state of the art as described above, it should probably therefore be seen as a technical problem, in a syringe in accordance with category “b” above, to be able to realise the importance of, the advantages associated with and/or the technical measures and considerations which will be required in order to cause to coupling devices, within the two-part coupling arrangement, to twist, by an axial displacement of a rod, out of an “active” position to an “inactive” position with slight friction and with slight torsional forces acting against the piston by causing said second coupling device to be related to means associated with a piston unit, and where means are, when necessary, to be rotatably coordinated with a piston included in said piston unit.

It should probably therefore be seen as a technical problem to be able to realise the importance of, the advantages associated with and/or the technical measures and considerations which will be required in order to cause said means to display a sub portion facing towards the piston and adapted for a rotary cooperation with a recess formed in the piston.

It should further probably be seen as a technical problem to be able to realise the importance of, the advantages associated with and/or the technical measures and considerations which will be required in order that said means be able to display a supporting sliding surface of low friction facing towards a sliding surface associated with the piston where in any event one of said sliding surfaces should be of planar configuration.

There resides a technical problem in being able to realise the importance of, the advantages associated with and/or the technical measures and considerations which will be required in order that said means be able to display a portion facing inwards towards the container with a sliding surface associated with the coupling device and given a configuration and curvature of the form of a cylindrical helix or conical helix.

There resides a technical problem in being able to realise the importance of, the advantages associated with and/or the technical measures and considerations which are required in order that the portion facing towards the means within the container display a support surface oriented transversely of a centre line to the means and the container.

There resides a technical problem in being able to realise the importance of, the advantages associated with and/or the technical measures and considerations which will be required in order to cause a support surface associated with said first coupling device to be given the configuration of a catch, with the support surface oriented transversely of a centre line to the means.

There resides a technical problem in being able to realise the importance of, the advantages associated with and/or the technical measures and considerations which will be required in order that said two support surfaces be mutually adapted with a total covering area so that, together and laterally oriented, they will form a surface extent which is less than the cross section of the container.

There resides a technical problem in being able to realise the importance of, the advantages associated with and/or the technical measures and considerations which will be required in order that, in an inactive position of the coupling devices, said two support surfaces are to be disposed laterally related and free from one another for a free passage of the support surface associated with the rod past the support surface associated with the piston unit.

There resides a technical problem in being able to realise the importance of, the advantages associated with and/or the technical measures and considerations which will be required in order to cause said sub portion associated with the means to be given spherical configuration.

Solution

The present invention thus takes as a point of departure the state of the art as disclosed by way of introduction for a syringe of non-reusable type, comprising: a container, a rod cooperating with the container, a piston unit inserted in and disposed to be reciprocal in the container, and a needle, the rod being, by the intermediary of an axial displacement movement, reciprocally moveable in said container and displaying in its one end portion enclosed by the container, a first coupling device within a two-part coupling arrangement whose second coupling device is related to said piston unit, and where said two coupling devices assume a mutually cooperating and active position while the piston unit is, by the movement of the rod, displaced from a position closely adjacent the needle to a position distal from the needle, and said coupling devices are gradually brought to an inactive position while the piston unit is, by the axial movement of the rod, displaced from the position distal from the needle towards and/or to the position closely adjacent the needle, the two coupling devices permitting, in an inactive position, an axial movement of the rod to take place without a cooperation with and corresponding displacement of the piston unit.

In order to be able to solve one or more of the above-outlined technical problems, the present invention particularly proposes that the prior art be supplemented by causing said second coupling device to have means coordinated with the piston within the piston unit which may be rotatably coordinated with said piston.

As preferred embodiments falling within the scope of the basic inventive concept of the present invention, it is proposed that said means display a sub portion adapted for cooperation with a recess in the piston and more particularly that said means display a supporting sliding surface facing towards a sliding surface to the piston.

In addition, it is proposed that in any event one of said sliding surfaces should be of planar configuration.

It is further proposed that said means display a portion facing inwards in the container with a sliding surface associated with the coupling device having been given a configuration and a curvature associating to a cylindrical helix and/or to a conical helix.

It is further proposed that the portion facing towards the means within the container display a support surface oriented transversely of a centre line to the means.

The support surface associated with said first coupling device has been given the configuration of a catch, oriented transversely of a centre line to the means.

More particularly, the present invention proposes that said coordinated support surfaces be adapted with a total covering area so that they, together and laterally coordinated, will form a surface extent which is but slightly less than a cross section to the container.

More particularly, according to the invention said support surfaces are, in said inactive position, caused to be disposed laterally related and free from one another for a free passage of the support surface associated with the rod past the support surface associated with the piston unit and the means.

The sub portion displaying said means should advantageously be given spherical configuration.

Advantages

The advantages which may principally be considered as characterising the present invention and the special significative characterising features thereby disclosed are that the prerequisites have hereby been created to be able to utilise a rod which, in a given axial displacement movement reciprocally within a container, creates the preconditions for a two-part coupling arrangement which only requires each one an opposed sliding surface, one for the first coupling device associated with the rod and one for the second coupling device associated with the piston unit, and to cause the second coupling device to be given means which advantageously and when necessary may be rotatably coordinated with a piston included in said piston unit and may thereby satisfy reliable demands that the two-part coupling arrangement assume an active position during a displacement movement of the rod from a fully inserted state to a withdrawn state, while the two-part coupling arrangement will bring said active coupling position to an inactive position while the piston unit and the rod are displaced from the position distal from the needle towards and/or to the position closely adjacent the needle and where the second coupling device associated with the piston unit is to be given means which, with low friction, may be rotatably coordinated with said piston unit.

That which may principally be deemed as characterising the present invention is disclosed in the characterising clause of appended claim 1.

### BRIEF DESCRIPTION OF THE ACCOMPANYING DRAWINGS

One currently proposed embodiment, displaying the significative characterising features associated with the present invention, will now be described in greater detail hereinbelow for purposes of exemplification and with reference to the accompanying Drawings. In the accompanying Drawings:

FIG. 1 shows, in side elevation, a syringe of non-reusable type comprising: a container, a rod cooperating with the container and moveably disposed by the intermediary of an “axial displacement movement”, a piston unit inserted in and reciprocally disposed in the container and with associated means and a needle; and

FIG. 2 shows, in a slightly perspective view, a two-part coupling arrangement according to the present invention with a first coupling device associated with the rod assuming a cooperating position with a second coupling device associated with the piston unit and associated means, and in which Figure arrows are employed to illustrate the movement required in order to rotate the second coupling device in relation to the first coupling device from an active to an inactive position.

### DESCRIPTION OF CURRENTLY PROPOSED EMBODIMENT

It should be emphasized by way of introduction that, in the following description of a currently proposed embodiment which displays the significative characterising features relating to the present invention and which is clarified by means of the Figures shown in the accompanying Drawings, we have selected terms and special terminology with the intention principally of clarifying the inventive concept.

However, in this context it should be observed that the expressions selected here should not be considered as restrictive exclusively to the terms selected and utilised here but it should be understood that each thus selected term is to be interpreted so that, in addition, it encompasses all technical equivalents which function in the same or substantially the same manner in order thereby to be able to attain the same or substantially the same intention and/or technical effect.

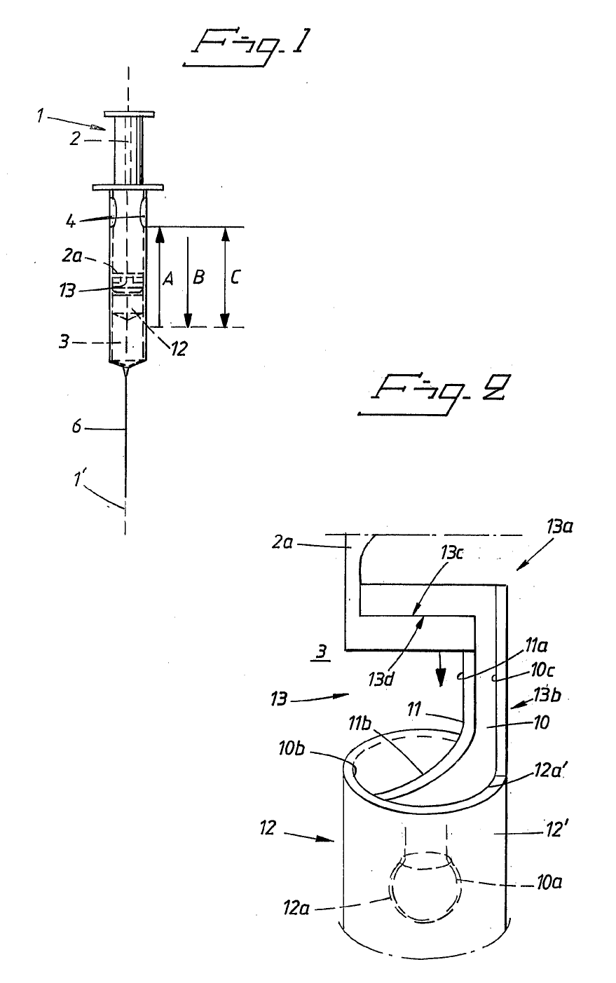

With reference to the FIG. 1, the basic preconditions for the present invention are thus shown schematically and where the significative properties associated with the invention have been rendered in generally concrete form as a result of the now proposed embodiment described in greater detail hereinbelow.

Thus, FIG. 1 shows in side elevation the basic construction of a syringe 1 of non-reusable type, comprising: a container 3, a rod 2 cooperating with the container, a piston unit 12 inserted in and, by the intermediary of an axial displacement movement, reciprocally disposed in the container 3, and a needle 6.

Without rotational movement, the rod 2 is axially displaceably disposed with the aid of means 4 reciprocally in said container 3.

With reference to FIG. 2, it is shown in greater detail that the rod, in its one end portion 2a enclosed by the container 3 cooperates a first connecting device 13a within a two-part coupling arrangement 13 whose second coupling device 13b is related to said piston unit 12.

Said two coupling devices 13a, 13b within said two-part coupling arrangement 13 initially assume a mutually cooperating and active position, according to FIG. 2, with the piston unit 12 in a position closely adjacent the needle 6 or wholly inserted position, and are retained in this cooperating position while the piston unit 12 is, by the movement of the rod 2, displaced from the position closely adjacent said needle to a position distal from the needle (where this displacement is illustrated in FIG. 1 by means of an arrow representing the selected direction of movement and the selected extent of movement “A”).

On an opposed direction of movement (the direction of movement and extent of movement “B”), the cooperation between the coupling devices 13a, 13b will gradually be brought to an inactive position, while the piston unit 12 is, by the movement of the rod 2, displaced from the position distal from the needle towards and/or to the position closely adjacent the needle.

This implies that, when the two coupling devices 13a, 13b assume an inactive position, the rod 2 may be given a reciprocally directed movement without the piston unit 12 accompanying this movement (the here intimated direction of movement and maximum extent of movement have been designated “C”), since the two coupling devices 13a, 13b are mutually rotated to an inactive position and may pass one another.

In this inactive and laterally displaced position of the coupling devices 13a and 13b, the syringe 1 is unusable for a replenishment of new liquid and subsequent injection of this new liquid.

In a construction of the type contemplated here there is required a rotary relative movement between the rod 2 and the piston unit 12 to twist the coupling devices 13a, 13b from an active to an inactive position and, since the rod 2 in this respect is non-rotational because of the edges 4, a rotational movement is required either for the entire piston unit 12 against the action of friction forces between the piston and the inner surface of the container and/or for means 10 against the action of friction forces between the means and the piston 12′.

With reference to FIG. 2, it is shown that said two-part coupling arrangement 13 assumes, with its first 13a and second 13b coupling device, an active and cooperating position and that a displacement of the rod 2 in the intimated direction “B” downwards in FIG. 1 will rotate the piston 12′ and/or means 10 associated with the piston unit 12 in relation to the rod 2 and/or the piston 12′ so that the two-part coupling arrangement 13 will assume an inactive position.

It will thus be apparent from FIG. 2 that said second coupling device 13b is to display or possess means 10 and where such means 10 may be rotatably coordinated with said piston 12′ included in the piston unit 12.

To this end, the embodiment according to FIG. 2 proposes that said means 10 display a sub portion 10a here adapted for a light rotary cooperation with a recess 12a in the piston unit 12.

In this instance, the sub portion 10a is formed in principle as a sphere adapted for light cooperation with a spheroid recess 12a in the piston 12′ and where said adaptation may very easily be lightly rotary.

More particularly, FIG. 2 shows that said means 10 may display a supporting sliding surface 10b facing towards the piston 12′ and where this support surface is to cooperate with and face towards a sliding surface 12a′ to the piston 12′, where in any event one of said sliding surfaces (10b or 12a′) should be of planar configuration.

There is nothing to prevent these sliding surfaces from having other configurations.

More particularly, FIG. 2 indicates that said means 10 may display a portion 10c facing inwardly towards the container 3, with a guide or sliding surface 11 associated with the coupling device 13b and given a configuration and a curvature associating to a cylindrical helix or to a conical helix.

More specifically, the sliding surface 11 is to display a first relatively short subsection 11a which is adapted so as to extend parallel to a centre line 1′ to the syringe 1 and along which subsection 11a the coupling devices 13a and 13b may move freely in relation to one another without any rotary movement being imparted to the piston unit 12 with the means 10 and the coupling device 13b, for an aspiration phase.

The sliding surface 11 also displays a second subsection 11b of curved configuration which, in conjunction with the previously disclosed planar sliding surface 10b, approximates to a cylindrical helix but merges, in a direction inwards in the container 3, into a more conical helix towards the lower region of the subsection 11a.

The subsections 11a and 11b are to be mutually adapted such that the coupling devices 13a and 13b are brought to their inactive position somewhat before the final displacement extent “B” is reached.

According to FIG. 2, the coupling devices 13a and 13b display, in their active position, mutually facing support surfaces, a first support surface associated with the rod and designated 13c and a second support surface associated with the piston unit and designated 13d.

More particularly, according to the invention the portion 10c of the means 10 facing inwardly in the container displays said support surface 13d, where this is to be oriented transversely of a centre line 1′ to the means 10 and the syringe 1.

The support surface 13c associated with said first coupling device 13a has been given the configuration of a catch, oriented transversely of a centre line 1′ to the means 10 and the syringe 1.

Said support surfaces 13c and 13d, respectively, are mutually adapted with a total covering area so that they together and laterally related to one another form a surface extent which approximates to but is only slightly less than a cross section of the container 3.

More particularly, it is disclosed that, in said wholly inactive position, said support surfaces 13c, 13d are to be disposed laterally related and free from one another for a free passage of the support surface 13c associated with the rod 2 past the support surface 13d associated with the piston unit 12 and the means 10.

The present invention is naturally not restricted to the embodiment disclosed by way of example above but may be subject to modifications without departing from the inventive concept as disclosed in the appended Claims.

In particular, it should be observed that each illustrated unit and/or circuit may be combined with every other shown unit and/or circuit without departing from the scope of the invention in order to be able to attain the desired technical function.

1. A syringe of non-reusable type comprising: a container, a rod cooperating with said container, a piston unit inserted in and reciprocally disposed in said container, and a needle, the rod being, by the intermediary of an axial displacement movement, reciprocally disposed in said container and displaying in its end portion, enclosed by the container, a first coupling device within a two-part coupling arrangement, whose second coupling device is related to said piston unit, and where said two coupling devices assume a mutually cooperation and an active position while the piston unit is, by the movement of the rod, displaced from a position, closely adjacent said needle, to a position distal from said needle and gradually brings said coupling devices towards and into an inactive position, while the piston unit is, by the movement of said rod, displaced from the position distal from the needle towards and/or to the position closely adjacent said needle, the two coupling devices permitting, in an inactive position, an axial movement of the rod to take place without cooperating with said piston unit, wherein, said second coupling device is provided with a piston unit related means, rotary coordinated with said piston within a piston unit, said unit displays a sub portion adapted for a rotary cooperation with a recess in said piston unit, said means displays a supporting sliding surface, facing towards a sliding surface provided on said piston unit where in any event one of said sliding surface is of a planar configuration, said means also displays a portion facing inwardly into the container with a sliding surface associated with the coupling device and given a configuration and a curvature associating to a cylindrical helix and/or to a conical helix, whereby said portion of the means facing inwardly into the container, displays a support surface, oriented transversely of a centre line related to said means, and that said support surface is cooperating with a further support surface, each adapted with a covering area to form surfaces which have a total area less than a cross section of said container and in said inactive position, said support surfaces are disposed laterally related and free from one another for a free passage of the support surface associated with the rod to pass the support surface associated with the piston.

2. A syringe as claimed in claim 1, wherein, said sub portion is given a spherical configuration.

3. A syringe as claimed in claim 1, wherein, a support associated with said first coupling device is in the form of a catch, oriented transversely of a centre line to said means.
